(19) United States
(12) Patent Application Publication
(10) Pub. No.: US 2011/0059025 A1
(43) Pub. Date: Mar. 10, 2011

(54) IN VIVO IMAGING

(75) Inventors: David Pritchard, Dundee (GB); Claire Geekie Dunn, Dundee (GB); Prabhjyot Dehal, Dundee (GB)

(73) Assignee: ITI SCOTLAND LIMITED, Glasgow, GB (GB)

(21) Appl. No.: 12/990,217

(22) PCT Filed: May 1, 2009
(86) PCT No.: PCT/EP2009/055320

§ 371 Date: Nov. 16, 2010

(30) Foreign Application Priority Data
May 2, 2008 (GB) 0808086.3

(51) Int. Cl. A61B 5/055

(52) U.S. Cl. 424/9.322; 424/9.34

(57) ABSTRACT

The present invention provides an imaging method for obtaining an image of a patient by means of a contrast agent wherein the method comprises subjecting the patient to an imaging method for which the contrast agent is suitable, wherein the contrast agent comprises: (a) a binding moiety; and (b) a recognition moiety capable of targeting the contrast agent to a site within the body of the patient, and wherein the binding moiety comprises a metal-binding protein, polypeptide or peptide which is bound to or encapsulates a magnetic or magnetizable substance.

### FIELD OF THE INVENTION

The present invention concerns the use of magnetic proteins, peptides and polypeptides in the field of in-vivo imaging. In particular, the invention provides a method of imaging utilising contrast agents, as well as contrast agent compositions to be used in such methods. The technology of the invention improves the detection, localisation and imaging of anatomical, physiological and pathological features in vivo, providing a cross-over between the fields of medical imaging and in vitro diagnostics.

### BACKGROUND OF THE INVENTION

One type of in vivo imaging is Magnetic Resonance Imaging (MRI), which is extensively used in the fields of veterinary and medical science. Most frequently it is used for the purpose of diagnosis but is also used to monitor the progress of therapy and for research purposes.

MRI makes use of the effect of nuclear magnetic resonance; the directional magnetic field (magnetic moment) that is associated with charge particles in motion. It allows images to be obtained in slices through a body so that a 3D picture can be created of internal structures within the body.

MRI images can be completed based on the content of the body alone. However, contrast agents are often administered to the patient prior to imaging to produce a better image and in particular to enhance the contrast in the image.

Several contrast agents are known in the art. Where the MRI is for imaging the stomach, for example, water may be used. Alternatively, contrast agents with magnetic properties can be used. One such example is the paramagnetic contrast agent, gadolinium. This agent provides highly sensitive detection of vascular tissues (e.g. tumours) and allows assessment of brain perfusion (e.g. in stroke patients). However, recently contrast agents based on gadolinium have been investigated for their toxicity, particularly to those patients suffering from impaired kidney function. These patients require haemodialysis after the MRI has been completed.

Despite this, gadolinium-based contrast agents have been the subject of several research methods. Choi et al., (Mol Imaging, 2007, 6(2): 75-84) describe the design of inflammation-targeted T(1) contrast agents prepared by bioconjugation of gandolinium diethylenetriaminepentaacetic acid (Gd-DTPA) with anti-intracellular adhesion molecule 1 (ICAM-1) antibody. The inflammation-specific T(1) enhancement was imaged with the Gd-DTPA-anti-ICAM-1 antibody in the mouse acute inflammation model.

Further, Artemov et al., (Cancer Research, 2003, 63: 2723-2727) describes a two-component gadolinium-based MR contrast agent system in which tumour cells expressing the HER-2/neu receptor are pre-labelled with biotinylated anti-HER2/neu antibody. Avidin-gandolinium complexes then specifically bind to the biotinylated receptors and positive T1 contrast in MR images was generated. The authors describe the use of this system with an experimental model of breast carcinomas derived from HER-2/neu transgenic mice.

Other examples of contrast agents are those which are superparamagnetic, e.g. iron oxide nanoparticles. These agents can be used to image the liver and the gastrointestinal tract.

There is an ongoing need to develop other contrast agents that provide alternatives to those currently available.

### SUMMARY OF THE INVENTION

Accordingly the present invention provides an imaging method for obtaining an image of a patient by means of a contrast agent wherein the method comprises subjecting the patient to an imaging method for which the contrast agent is suitable, wherein the contrast agent comprises:
(a) a binding moiety; and
(b) a recognition moiety capable of targeting the contrast agent to a site within the body of the patient,
   and wherein the binding moiety comprises a metal-binding protein, polypeptide or peptide which is bound to or encapsulates a magnetic or magnetizable substance.

The inventors have surprisingly discovered that the contrast agent defined above can be utilised in an imaging method to enhance the contrast of the images obtained. In particular, the contrast agents contain a recognition moiety capable of targeting the contrast agent to a site within the body of the patient. This allows the targeting of the contrast agent after its administration to the patient to specific site or sites of interest. Areas where localisation of the magnetic substance occurs are visualised with in vivo imaging. In contrast the methods of the prior art utilised contrast agents which are non-specific and do not have this biological recognition function.

A particular advantage of a method of the present invention is that it expands the amount of information that can be obtained from the images generated. In particular, it provides the possibility of obtaining additional and more specific diagnostic information from the images generated. For example, using a contrast agent with a recognition moiety capable of binding to a specific type of tumour cell will provide additional information on the nature of any tumour visible in the image generated, and may aid the production of higher resolution image.

The present invention also provides contrast agents that are suitable for use in the method of the invention. In particular, the present invention provides a contrast agent composition suitable for use in an imaging method wherein the agent comprises:
(a) a binding moiety; and
(b) a recognition moiety;
   wherein the binding moiety comprises a metal-binding protein, polypeptide or peptide which is bound to or encapsulates a magnetic or magnetizable substance, and wherein the composition optionally contains a further component suitable for use in a contrast agent composition.

The present invention also provides for the use of a contrast agent in method for obtaining an image of a patient wherein the agent comprises:
(a) a binding moiety; and
(b) a recognition moiety;
   and wherein the binding moiety comprises a metal-binding protein, polypeptide or peptide which is bound to or encapsulates a magnetic or magnetizable substance.

Additional advantages of the contrast agent described herein stem from the magnetic or magnetizable substance which it contains. In particular, the contrast agent can be administered to and removed from a site within the body of the patient using a device comprising an electromagnet and an element suitable for bringing the contrast agent into proximity with the site, or by dialysis.

Still further, the magnetic properties of the contrast agent facilitate its production and purification. In particular, the agents are simple to purify using established techniques, such as affinity purification, or magnetic field purification. The contrast agents of the present invention have the advantage that they may be magnetised or de-magnetised using simple chemical procedures.

### DETAILED DESCRIPTION OF THE INVENTION

The invention will now be described in further detail with reference, by way of example only, to the accompanying drawing.

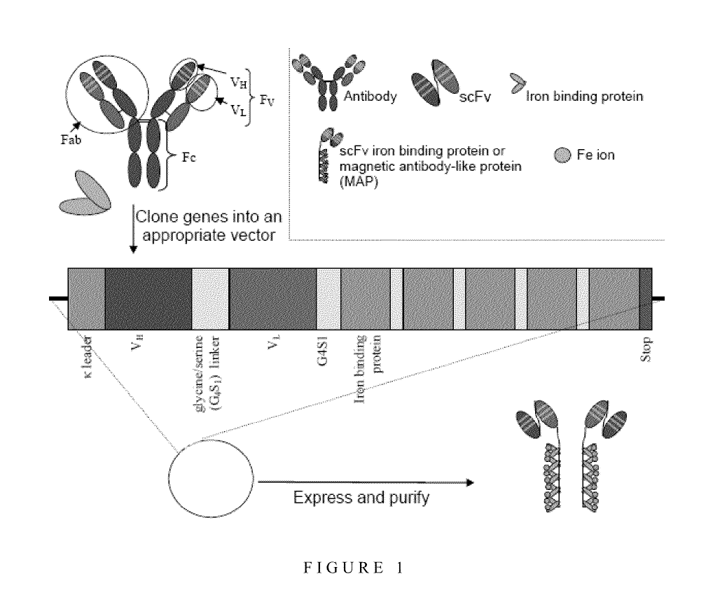

FIG. 1: this Figure shows how the appropriate genes are cloned into a vector in order to produce the contrast agents of the present invention. The number of magnetizable protein units in the final contrast agent may be controlled by including as many copies of the appropriate gene as necessary. Only genes for the V_{H} and V_{L} regions of the antibody are included in this example, so that the scFv portion of the antibody is included in the final preferred chimaeric protein, rather than the full antibody.

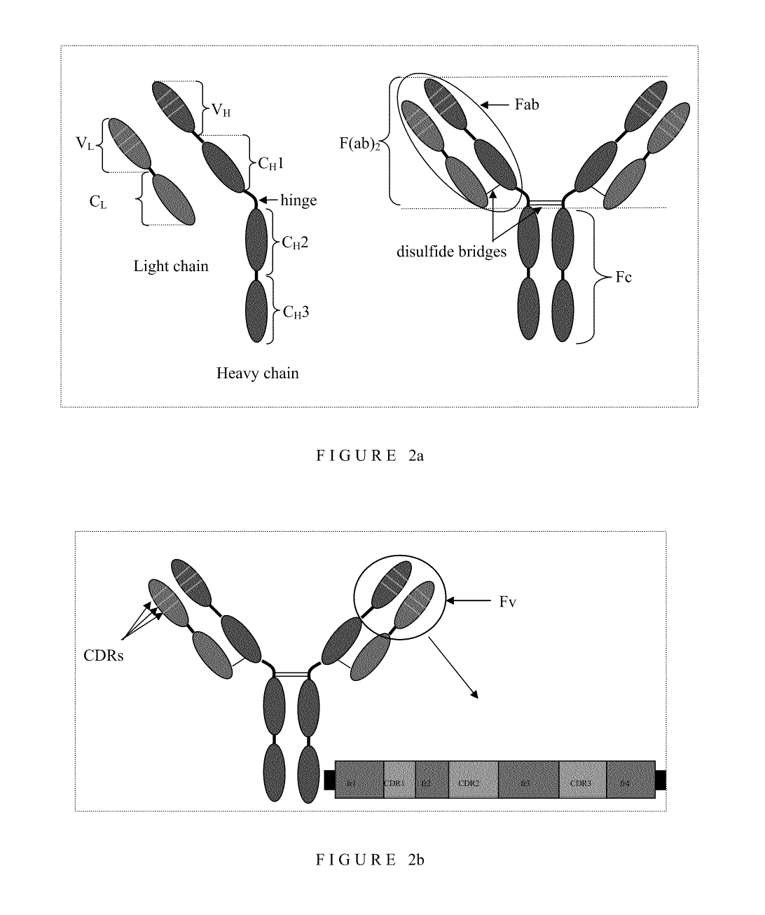

FIGS. 2a and 2b: these Figures schematically depict a simplification of the structure of antibodies such as IgG. After protease treatment using enzymes such as papain, antibodies are split into 3 parts close to the hinge region. As the effector function part of antibodies (the hinge, C_{H}2 and C_{H}3) are relatively easy to crystallise for X-ray diffraction analysis, this part has become known as the crystallisable fragment (Fc) region. The antigen binding portions of antibodies are known as the antibody fragment (Fab). After enzymic digestion, the Fab fragments can be linked at the hinge region thereby forming a F(ab)₂ fragment. Other antibodies may have differences in the number of domains in the Fc region and variations in the hinge region.

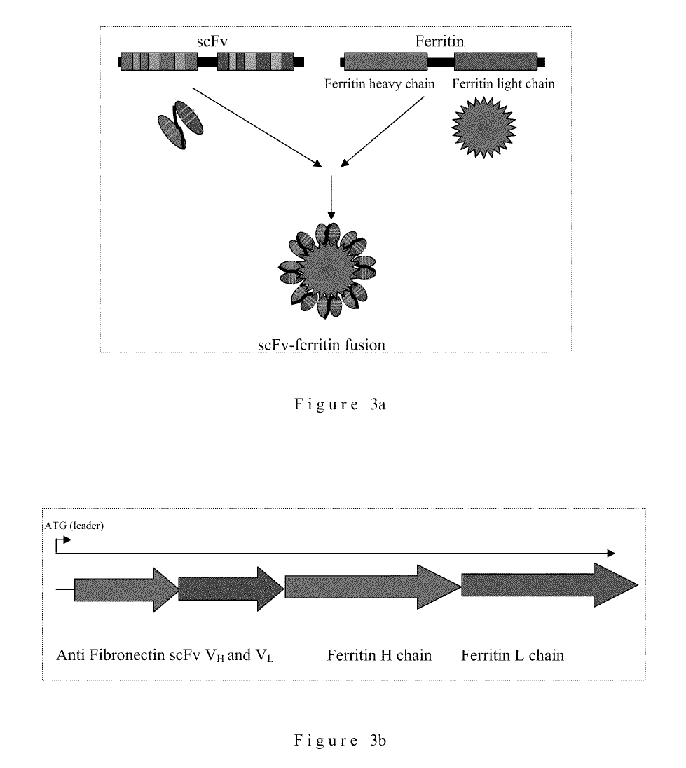

FIGS. 3a and 3b: these Figures show the construction of a scFv-ferritin fusion protein.

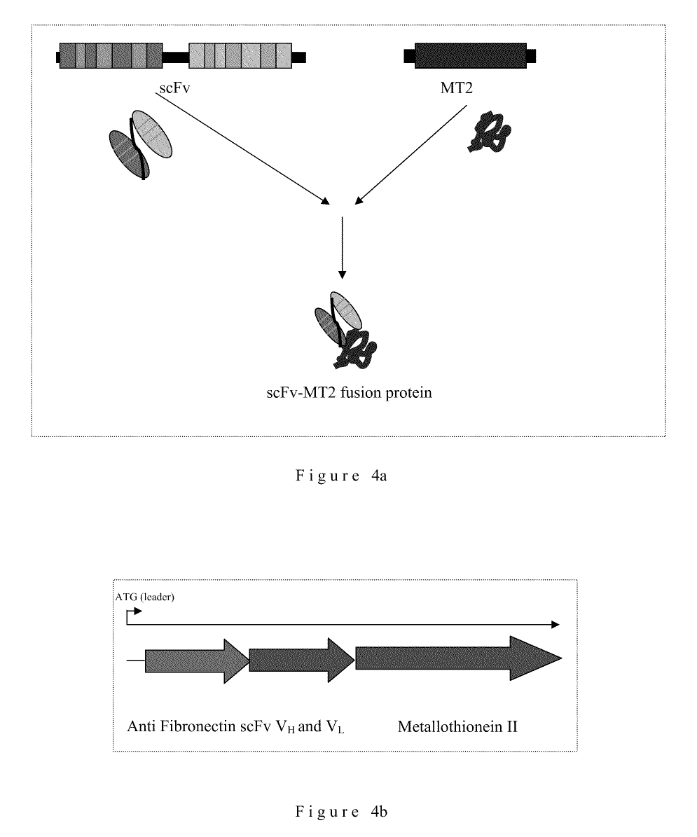

FIGS. 4a and 4b: these Figures show the construction of a scFv-MT2 fusion protein.

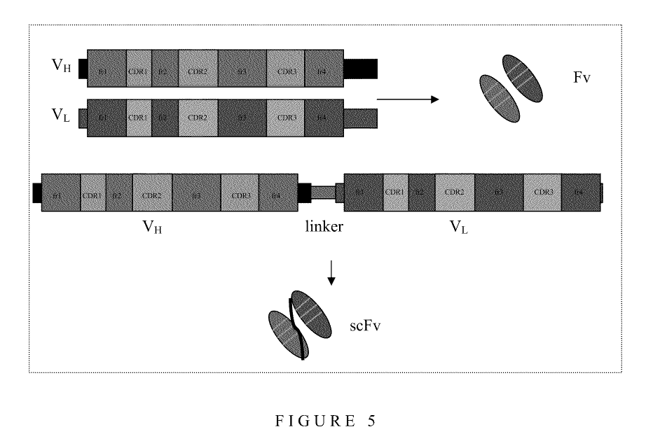

FIG. 5: this Figure shows the construction of a scFv fragment.

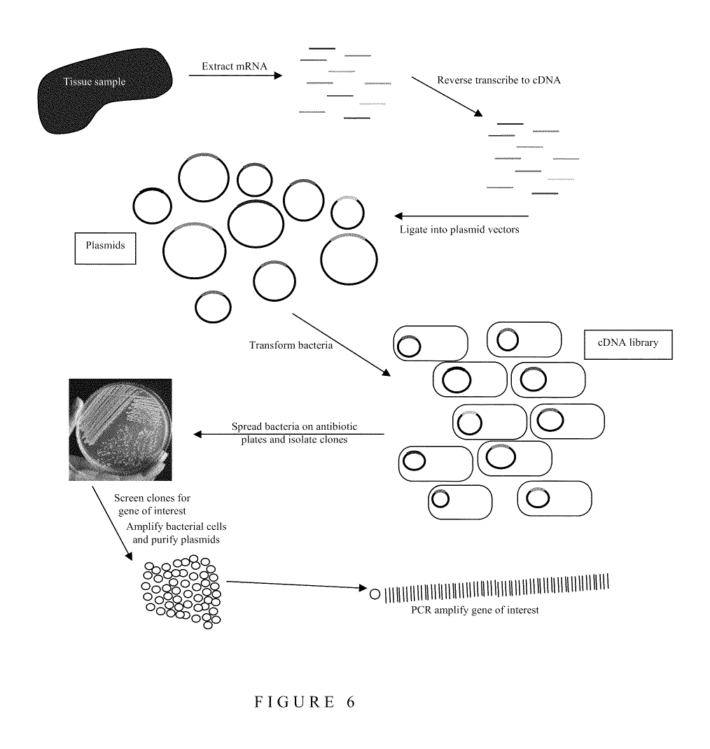

FIG. 6: this Figure shows construction of a cDNA library. In order to construct a cDNA library from a tissue sample, mRNA is extracted, reverse transcribed into cDNA and ligated into plasmid vectors. These vectors are then used to transform bacteria cells. The transformed cells are stored frozen until required. The frozen cells can be expanded by growing in appropriate media and the plasmids purified. Genes of interest can then be PCR amplified for further analysis using specific primer pairs.

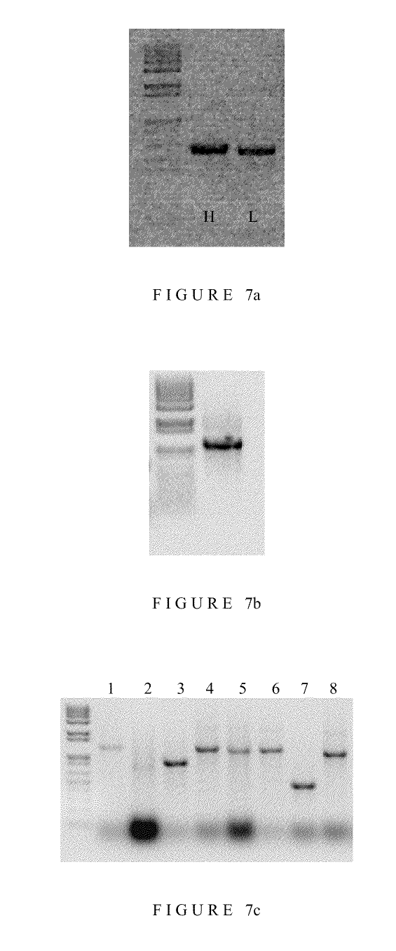

FIGS. 7a and 7b: these Figures show PCR amplicons of the ferritin heavy (H) and light (L) chain genes, and the overlapped PCR product of ferritin heavy and light chain genes, respectively. FIG. 7c shows colony PCR results, clones 1, 3 and 4 were selected for sequencing.

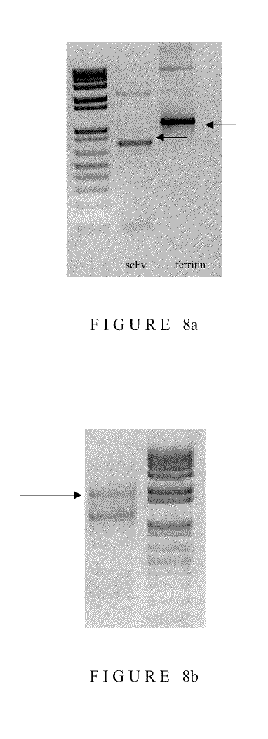

FIGS. 8a and 8b: these Figures show a gel showing the products of a PCR amplification of the anti-fibronectin scFv and ferritin heavy and light polygene (arrowed), and a gel showing the overlap PCR products, respectively.

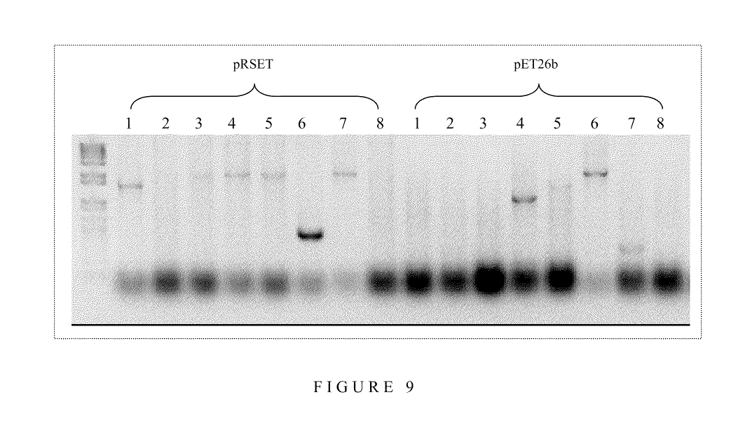

FIG. 9: this Figure shows a gel showing the results of a PCR screen of a number of clones transformed using plasmids that had been ligated with the scFv:ferritin fusion constructs.

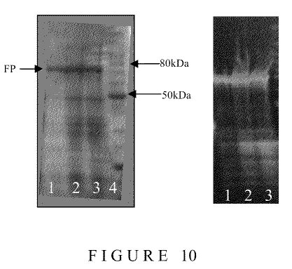

FIG. 10: this Figure shows Coomassie blue stained gel and Western blot of cell lysates respectively. Key: 1. Ferritin 2 hour induction; 2. Ferritin 3 hour induction; 3. Ferritin 4 hour induction; 4. Benchmark (Invitrogen) Protein Ladder.

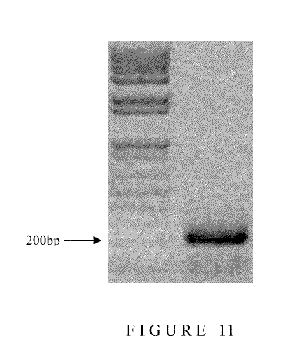

FIG. 11: this Figure shows a gel showing the PCR amplification product of MT2 from a human liver library.

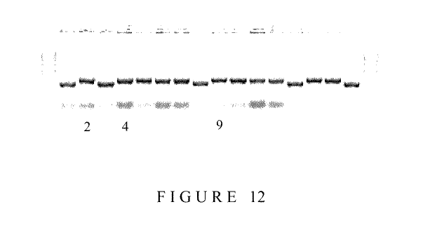

FIG. 12: this Figure shows colony analysis of clones transformed with plasmid containing the scFv:MT2 construct.

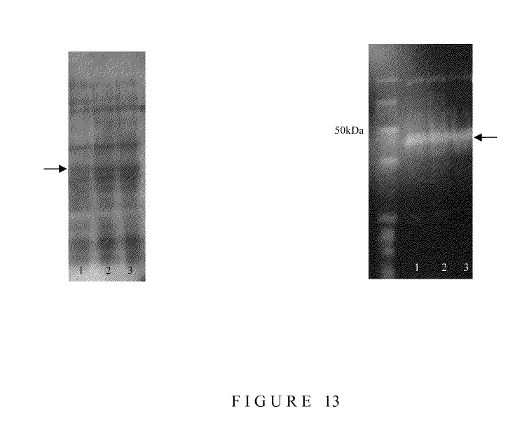

FIG. 13: this Figure shows (respectively) Coomassie gel and western blot of scFv:MT2 (arrowed).

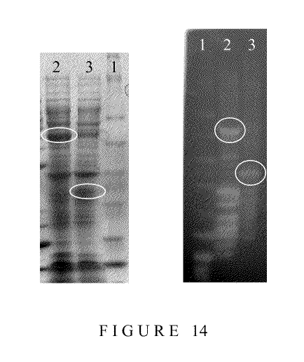

FIG. 14: this Figure shows photographs of a Coomassie blue stained gel and western blot (respectively) of the re-solubilised scFv:ferritin and scFv:MT2 fusion proteins. The fusion proteins are circled—ferritin is in lane 2 on both gels and MT2 is in lane 3 of both gels. A protein molecular weight ladder is in lane 1.

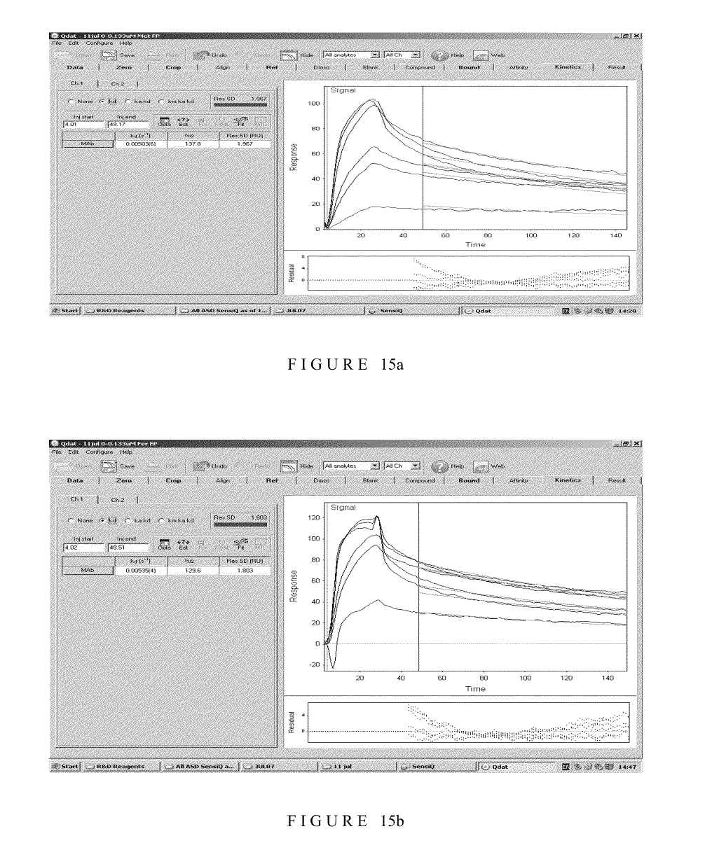

FIGS. 15a and 15b: these Figures show overlaid Sensograms from the SPR analysis of the binding of MT2 and ferritin fusion proteins respectively.

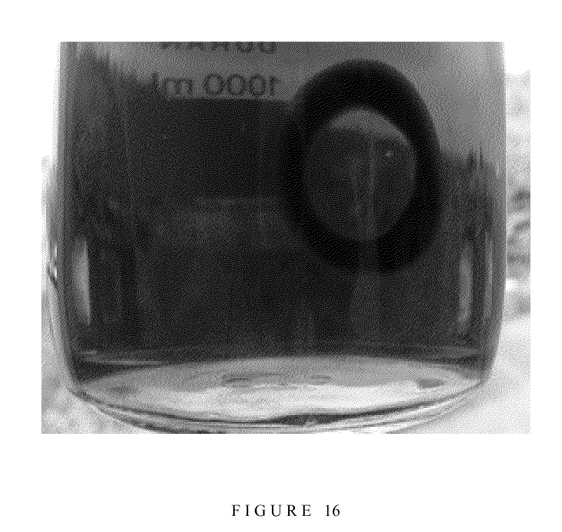

FIG. 16: this Figure demonstrates the magnetic nature of the magnetoferritin produced for use in the present invention.

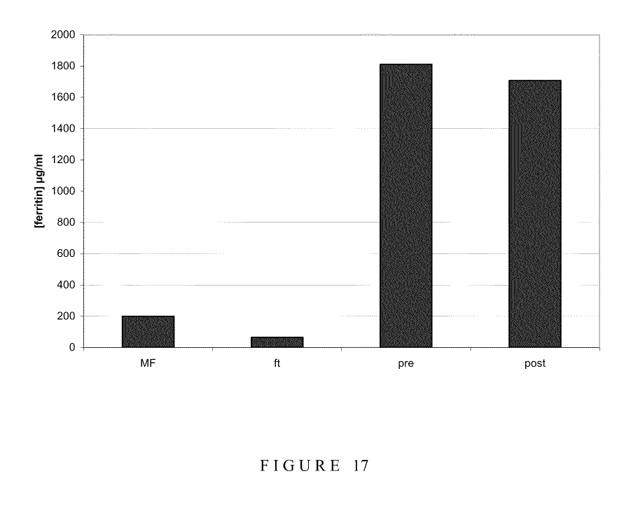

FIG. 17: this Figure shows the concentration of ferritin during the production and concentration of magnetoferritin. Key: MF; Magnetoferritin: ft; Flow-through: Pre; pre-dialysis Macs® column concentrated magnetoferritin: post; post-dialysis Macs® column concentrated magnetoferritin.

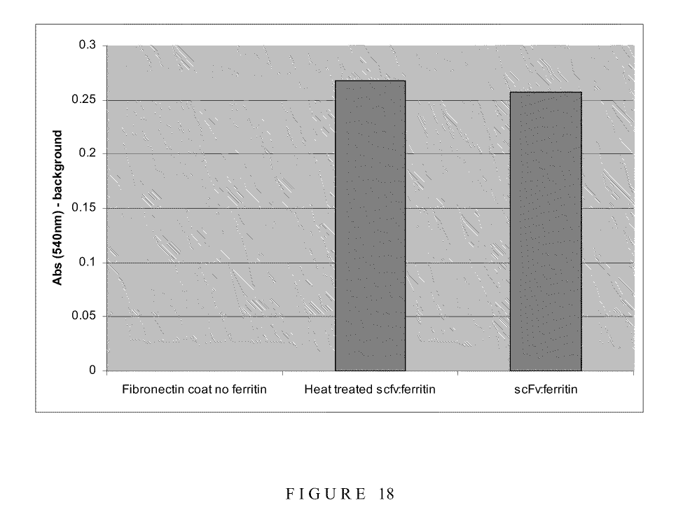

FIG. 18: this Figure shows binding of scFv:ferritin and heat treated scFv:ferritin to fibronectin.

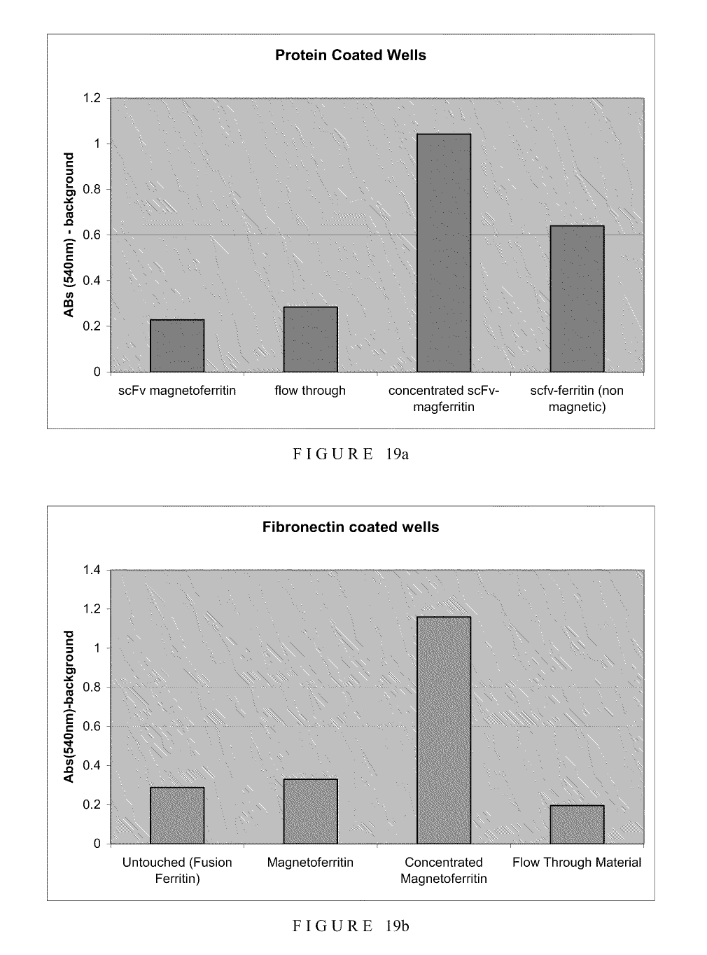

FIGS. 19a and 19b: these Figures show absorbance measurements, recorded using a Varioskan Flash instrument, on magnetised fusion protein. After concentration the protein is still recognised by the monoclonal anti-ferritin antibody (19a) and the magnetised anti-fibronectin ferritin fusion protein retains binding ability to its target antigen (19b).

As described above, the present invention relates to an imaging method for obtaining an image of a patient by means of a contrast agent wherein the method comprises subjecting the patient to an imaging method for which the contrast agent is suitable, wherein the contrast agent comprises:
(a) a binding moiety; and
(b) a recognition moiety capable of targeting the contrast agent to a site within the body of the patient,
   and wherein the binding moiety comprises a metal-binding protein, polypeptide or peptide which is bound to or encapsulates a magnetic or magnetizable substance.

In one aspect of the present invention the method further comprises a step of administering the contrast agent to the patient.

The imaging method of the present invention may be a magnetic resonance imaging (MRI). It may also be a method of nuclear magnetic resonance (NMR) or a method of electron spin resonance (ESR).

In a preferred aspect of the invention the imaging method is magnetic resonance imaging. Methods of magnetic resonance imaging are well known in the art. Usually they comprise the steps of administering a contrast agent to a patient, positioning the patient in a magnetic resonance imaging system, and using the system to obtain at least one image of the patient's body. Commonly used magnetic field strengths range from 0.3 to 3 teslas. However, the method of the present invention can be used across the full range of field strengths applied in the art, i.e. up to approximately 20 teslas.

Similarly, the method of the present invention can be utilised with other specific MRI techniques, such as Diffusion Weighted Imaging (DWI).

The binding moiety which binds to or encapsulates the magnetic or magnetizable substance is not especially limited, provided that it is non-toxic, capable of binding the substance and capable of being attached to the recognition moiety. The binding moiety comprises a metal-binding protein, polypeptide or peptide (or the metal-binding domain of such a protein polypeptide or peptide). The binding moiety is be capable of binding or encapsulating (or otherwise attaching in a specific or non-specific manner) to the magnetic or magnetizable substance in the form of particles or aggregates or the like.

These particles or aggregates typically have less than 100,000 atoms, ions or molecules, more preferably less than 10,000 atoms, ions or molecules, and most preferably less than 5,000 atoms ions or molecules bound or encapsulated to the (or each) moiety in total. The most preferred substances are capable of binding up to 3,000 atoms ions or molecules, and in particular approximately 2,000 or less, or 500 or less such species.

In one specific example employed in the invention, the binding moiety comprises the metallic component of ferritin (a 24 subunit protein shell) consists of an 8 nm (8×10⁻⁹ m) inorganic core. Each core contains approximately 2,000 Fe atoms. In another example, Dpr, from Streptococcus mutans (a 12 subunit shell), consists of a 9 nm shell containing 480 Fe atoms. In a further example, lactoferrin binds 2 Fe atoms and contains iron bound to haem (as opposed to ferritin which binds iron molecules within its core). Metallothionein-2 (MT) binds 7 divalent transition metals. The zinc ions within MT are replaced with Mn²⁺ and Cd²⁺ to create a room temperature magnetic protein. MT may be modified to further incorporate one or more additional metal binding sites, which increases the magnetism of the Mn, Cd MT protein.

In accordance with these binding environments, the total volume of the substance bound or encapsulated in a single moiety typically does not exceed 1×10⁵ nm³ (representing a particle or aggregate of the substance having an average of about 58 nm or less). More preferably the substance may have a total volume of not more than 1×10⁴ nm³ (representing a particle or aggregate of the substance having an average diameter of about 27 nm or less). More preferably still the substance may have a total volume of not more than 1×10³ nm³ (representing a particle or aggregate of the substance having an average diameter of about 13 nm or less). Most preferably the substance may have a total volume of not more than 100 nm³ (representing a particle or aggregate of the substance having an average diameter of 6 nm or less). However, the size of the particles may be determined by average diameter as an alternative to volume. It is thus also preferred in the present invention that the average diameter of the bound particles is 50 nm or less, 40 nm or less, 30 nm or less, 20 nm or less or most preferably 10 nm or less. In this context, average means the sum of the diameters of the number of particles, divided by the number of particles.

In a particularly preferred embodiment of the invention the magnetic or magnetizable substance is paramagnetic and only exhibits magnetism under the influence of a stronger magnet. The advantage of the use of a paramagnetic substance is that any clumping of the contrast agent is avoided until the scan.

Typically the binding moiety is bound to or encapsulates one or more transition and/or lanthanide metal atoms and/or ions, or any compound comprising such ions. Such ions include, but are not limited to, any one or more ions of Fe, Co, Ni, Mn, Cr, Cu, Zn, Cd, Y, Gd, Dy, or Eu.

In the more preferred embodiments of the invention, the one or more metal ions comprise any one or more of Fe²⁺, Fe³⁺, Co²⁺, Co³⁺, Mn²⁺, Mn³⁺, Mn⁴⁺, Cd²⁺, Zn²⁺, Gd³⁺ and Ni²⁺. The most preferred ions for use in the present invention are Fe²⁺, Fe³⁺, Cd²⁺, Mn²⁺, Gd³⁺, Co²⁺ and Co³⁺ ions. Typically these ions are bound by lactoferrin, transferrin and ferritin in the case of iron, and metallothionein-2 in the case of cadmium and manganese. The binding of Fe²⁺ is preferably promoted by employing acidic conditions, whilst the binding of Fe³⁺ is preferably promoted by employing neutral or alkaline conditions.

In preferred embodiments of the invention, the metal-binding moiety comprises a protein, or a metal-binding domain of a protein, selected from lactoferrin, transferrin, ferritin (apoferritin), a metallothionein (MT1 or MT2), a ferric ion binding protein (FBP e.g. from Haemophilus influenzae), frataxin and siderophores (very small peptides which function to transport iron across bacterial membranes).

### Metal Binding Proteins

The number of metal binding proteins described in the literature is still increasing. Many proteins store iron (Fe) as an oxyhydroxide-ferric phosphate or as haem, therefore complicating magnetising methods. Proteins such as ferritin are able to store thousands of iron ions within a cage-like structure.

As the endogenous iron within ferritin is not paramagnetic, it typically needs to be removed and replaced with a paramagnetic form without damaging the protein. Other metal binding proteins such as metallothionein II (MT2) hold fewer ions of metal in a loose lattice arrangement, and it may be easier to remove and replace these than with ferritin.

### Ferritin

Ferritin is a large protein, 12-nm diameter, with a molecular weight of 480 kDa. The protein consists of a large cavity (8 nm diameter) which encases iron. The cavity is formed by the spontaneous assembly of 24 ferritin polypeptides folded into four-helix bundles held by non-covalent bonds. Iron and oxygen form insoluble rust and soluble radicals under physiological conditions. The solubility of the iron ion is 10⁻¹⁸M. Ferritin is able to store iron ions within cells at a concentration of 10⁻⁴M.

The amino acid sequence, and therefore the secondary and tertiary structures of ferritin are conserved between animals and plants. The sequence varies from that found in bacteria; however, the structure of the protein in bacteria does not. Ferritin has an essential role for survival as studies using gene deletion mutant mice resulted in embryonic death. Ferritin has also been discovered in anaerobic bacteria.

Ferritin is a large multifunctional protein with eight Fe transport pores, 12 mineral nucleation sites and up to 24 oxidase sites that produce mineral precursors from ferrous iron and oxygen. Two types of subunits (heavy chain (H) and light chain (L)) form ferritin in vertebrates, each with catalytically active (H) or inactive (L) oxidase sites. The ratio of heavy and light chains varies according to requirements. Up to 4000 iron atoms can be localised in the centre of the ferritin protein.

The iron stored within ferritin is usually in the form of hydrated iron oxide ferrihydrite (5Fe₂O₃.9H₂O). It is possible to replace the ferrihydrite core with ferrimagnetic iron oxide, magnetite (Fe₃O₄). This may be achieved by removing the iron using thioglycolic acid to produce apoferritin. Fe(II) solution is then gradually added under argon or other inert gas with slow, controlled oxidation by the introduction of air, or an alternative oxidising agent.

### Metallothionein II

Metallothioneins are intracellular, low molecular weight, cysteine-rich proteins. These proteins are found in all eukaryotes and have potent metal-binding and redox capabilities. MT-1 and MT-2 are rapidly induced in the liver by a variety of metals, drugs and inflammatory mediators. The functions of MT-2 include zinc (Zn) homeostasis, protection from heavy metals (especially cadmium) and oxidant damage and metabolic regulation.

MT2 binds seven divalent transition metals via two metal binding clusters at the carboxyl α-domain) and amino (β-domain) terminals. Twenty cysteine residues are involved in the binding process.

Chang et al describe a method of replacing the seven zinc (Zn²⁺) ions with manganese (Mn²⁺) and cadmium (Cd²⁺) ions. The resultant protein was shown to exhibit a magnetic hysteresis loop at room temperature. This could potentially mean that the protein is paramagnetic.

Toyama et al engineered human MT2 to construct an additional metal binding site. This could potentially increase the paramagnetic functioning of the MT2, and may be employed in the present invention.

In some embodiments, the contrast agent of the invention may comprise a plurality of binding moieties binding or encapsulating the magnetic or magnetizable substance. The number of such moieties may be controlled so as to control the magnetic properties of the contrast agent. Typically in such embodiments, the contrast agent may comprise from 2-100 such moieties, preferably from 2-50 such moieties and most preferably from 2-20 such moieties for binding the magnetic or magnetizable substance. In the final chimaeric protein, each copy of the metal-binding protein may be attached to the next by non-charged amino acid linker sequences for flexibility.

In further embodiments modifications such as glycosylation or phosphorylation may be made to the protein/peptide binding moieties so as to adjust their (electro)magnetic properties.

In the method of the present invention the contrast agent also comprises a recognition moiety which is capable of binding to a target within the body of the patient. Examples of possible targets are carcinomas/tumours, cysts (such as endometriosis cysts), benign growths, cardiovascular plaques, neurological plaques (such as those found in Alzheimer's Disease, neurofibrillary tangles, and β-amyloid plaques), areas of the body undergoing angiogenesis, areas of the body undergoing apoptosis and necrosis, thrombi, areas of inflammation, e.g. in rheumatoid arthritis and diabetes, and areas of the body which are infected with, for example, infectious diseases such as bacterial/fungal infections. In particular, the method of the present invention is able to image small and difficult to detect carcinomas, and secondary tumours at an early stage in their development, which are not detectable by other methods. Intracellular targeting is also possible. A nuclear localisation signal can be used as recognition moiety to target the contrast agent to the nucleus. Alternatively, recognition moieties can be selected to target the contrast agent to the Golgi apparatus or the inner cell membrane.

In particular, the recognition moiety may recognise an antigen expressed on the surface of a tumour cell. Some tumours express a variety of antigens on their surface. Accordingly, it is particularly preferred that the vector comprises at least two recognition moieties which recognise and bind at least two different antigens on the surface of a tumour cell. In an alternative arrangement the vector comprises at least two recognition moieties to achieve receptor cross-linking and internalisation of the complex.

The recognition moiety that is capable of binding to the above targets may itself be any type of substance or molecule, provided that it is suitable for binding to the target. Generally, the recognition moiety is selected from an antibody or a fragment of an antibody, a receptor or a fragment of a receptor, a protein, a polypeptide, a peptidomimetic, a nucleic acid, an oligonucleotide and an aptamer. In more preferred embodiments of the invention, the recognition moiety is selected from a variable polypeptide chain of an antibody (Fv), a T-cell receptor or a fragment of a T-cell receptor, avidin, and streptavidin. Most preferably, the recognition moiety is selected from a single chain of a variable portion of an antibody (sc-Fv).

Antibodies are immunoglobulin molecules involved in the recognition of foreign antigens and expressed by vertebrates. Antibodies are produced by a specialised cell type known as a B-lymphocyte or a B-cell. An individual B-cell produces only one kind of antibody, which targets a single epitope. When a B-cell encounters an antigen it recognises, it divides and differentiates into an antibody producing cell (or plasma cell).

The basic structure of most antibodies is composed of four polypeptide chains of two distinct types (FIG. 2). The smaller (light) chain being of molecular mass 25 kilo-Daltons (kDa) and a larger (heavy) chain of molecular mass 50-70 kDa. The light chains have one variable (V_{L}) and one constant (C_{L}) region. The heavy chains have one variable (V_{H}) and between 3-4 constant (C_{H}) regions depending on the class of antibody. The first and second constant regions on the heavy chain are separated by a hinge region of variable length. Two heavy chains are linked together at the hinge region via disulfide bridges. The heavy chain regions after the hinge are also known as the Fc region (crystallisable fragment). The light chain and heavy chain complex before the hinge is known as the Fab (antibody fragment) region, with the two antibody binding sites together known as the F(ab)₂ region. The constant regions of the heavy chain are able to bind other components of the immune system including molecules of the complement cascade and antibody receptors on cell surfaces. The heavy and light chains of antibodies form a complex often linked by a disulfide bridge, which at the variable end is able to bind a given epitope (FIG. 2).

The variable genes of antibodies are formed by mutation, somatic recombination (also known as gene shuffling), gene conversion and nucleotide addition events.

The antigen binding portions of antibodies can be used in isolation without the constant regions. This may be of some use in, for example, designing recognition moieties better adapted to penetrate solid tumours. The V_{H} and V_{L} domains can be expressed in cells as an Fv fragment. Alternatively, the two domains can be linked by a short chain of small amino acids to form a single polypeptide known as a single chain Fv fragment (scFv), which has a molecular weight of approximately 25 KDa (see FIG. 5). The linker is composed of a small number of amino acids such as serine and glycine which do not interfere with the binding and scaffold regions of the scFV.

ScFv antibodies may be generated against a vast number of targets including:
1. Viruses: Torrance et al. 2006. Oriented immobilisation of engineered single-chain antibodies to develop biosensors for virus detection. J Virol Methods. 134 (1-2) 164-70.
2. Hepatitis C virus: Gal-Tanamy et al. 2005. HCV NS3 serine protease-neutralizing single-chain antibodies isolated by a novel genetic screen. J Mol. Biol. 347 (5):991-1003), and Li and Allain. 2005 Chimeric monoclonal antibodies to hypervariable region 1 of hepatitis C virus. J Gen Virol. 86 (6) 1709-16.
3. Cancers: Holliger and Hudson. Engineered antibody fragments and the rise of single domains. Nat. Biotechnol. 23 (9) 1126-36.

Within the contrast agent the recognition moiety is attached to the binding moiety. By ‘attached to’ in the present context it is meant that the attachment is of any type including specific and non-specific binding, and also covers encapsulation of the binding moiety by the recognition moiety.

In a particularly preferred aspect of the present invention the binding moiety and the recognition moiety are attached in the form of a fusion protein. In the context of the present invention, a fusion protein is a protein that has been expressed as a single entity recombinant protein. The use of fusion proteins in the vector creates a number of further advantages. The orientation of the recognition arm of the fusion protein (e.g. the scFv) within the agent will be controlled and therefore more likely to bind its target. Fusion proteins also facilitate the possibility of incorporating a plurality of recognition moieties in a single fusion protein. These recognition sites may be directed against the same target or to different targets. Where two or more recognition moieties are present, the spatial organisation of the recognition moieties on the magnetic substance can be defined and controlled, decreasing problems caused by steric hindrance and random binding. With careful spacing of each recognition moiety within the fusion protein (e.g. by incorporating nucleic acid spacers in the expression system) the tertiary structure of the final protein can be controlled to deploy recognition moieties at spatially selected zones across the protein surface. It is preferred that the binding moiety and the recognition moiety in the fusion protein are separated by a linker. The linker is typically less than 15 amino acid, preferably less than 10 amino acids and most preferably less than 5 amino acids in length. A further advantage of using fusion proteins is that the number of recognition moieties within each particle of the agent can be specified and will be identical for every molecule of the agent.

Moreover, where the binding moiety is made up of several subunits which assemble together to form a particle, different subunits can be utilised. In this way a heterogeneous particle can be obtained in which some subunits are attached to a recognition moiety, while others are not. The number of recognition moieties comprised in the contrast agent can therefore be controlled by using different ratios of subunits. These particles can be generated to avoid problems of steric hindrance and achieve more efficient binding between the contrast agent and the target.

An embodiment of the invention which utilises fusion proteins is one in which the contrast agent comprises a binding moiety which is a plurality of ferritin subunits, which assemble to form a particle, with the recognition moieties present on the outer surface thereof. Such a particle may encapsulate magnetic or magnetizable material.

The contrast agents of the present invention may optionally incorporate specific cleavage sites between the binding moiety and the recognition moiety, within the recognition moiety or, where the binding moiety is an assembled particle, between subunits of the particle, so as to allow the contrast agent to be broken down if required. This can particularly be achieved by incorporating specific protease cleavage sites into the contrast agent.

For example, the subunits of the binding moiety can be linked by a length of amino acid residues which provide a cleavage site for a specific protease. During use, when the contrast agent is exposed to the protease, it will be broken down, thus releasing the encapsulated magnetic or magnetizable substance. Specific cleavage sites can be used which are only recognised in particular cell types or tissues, leading to selective breakdown. Alternatively, the cleavage site may be within the recognition moiety such that action by a protease can remove the upper segment of the recognition moiety to “reveal” a second recognition moiety with different specificity.

### Fusion Protein Design

In the present invention, the fusion proteins may be designed using the variable regions from an anti-fibronectin murine monoclonal IgG1 antibody to generate a scFv domain. The heavy and light chains of ferritin or the MT2 gene can be used to generate the magnetic domain of the antibody. The genes for the variable domains of the anti-fibronectin antibody are commercially available, and these are typically cloned into a plasmid vector to be expressed as a scFv. The scFv may be translated in the following order:
ATG start codon: leader sequence (for expression): heavy chain: glycine serine linker: light chain.

### Plasmid Generation

The genes for the human heavy and light chains of ferritin or human MT2 may be obtained from a human library, cloned using appropriately designed primers and inserted into the anti-fibronectin scFv plasmid vector at the 3′ end of the antibody light chain with a terminal stop codon. Genes fused to the 3′ end of the heavy and light chains of ferritin may be expressed within the ferritin molecule rather than on the surface. Therefore, the scFv ferritin fusion construct has the scFv at the N-terminal (corresponding to the 5′ end) of the ferritin heavy chain. The scFv and ferritin or MT2 fusion proteins typically have a histidine tag (consisting of six histidine residues) at the C-terminus of the protein before the stop codon. This allows for the detection of the proteins in applications such as Western blotting, and for possible purification using metal affinity columns (such as nickel columns) or other tags (e.g. GST, β-galactosidase, HA, GFP) if the metal binding functions interfere. The sequences of the genes may be checked after plasmid production to ensure no mutations had been introduced.

FIG. 3b is a diagrammatic representation of an exemplary ferritin fusion protein. The scFv heavy and light chains are represented by first two arrows respectively.

The sequence employed was SEQ ID 1 set out below:

SEQ ID No: 1
LVQPGGSLRLSCAASGFTFSSFSMSWVRQAPGKGLEWVSSISGSSGTTYY
ADSVKGRFTISRDNSKNTLYLQMNSLRAEDTAVYYCAKPFPYFDYWGQGT
LVTVSSGDgssggsggASTGEIVLTQSPGTLSLSPGERATLSCRASQSVS
SSFLAWYQQKPGQAPRLLIYYASSRATGIPDRFSGSGSGTDFTLTISRLE
PEDFAVYYCQQTGRIPPTFGQGTKVEIKsgggMTTASTSQVRQNYHQDSE
AAINRQINLELYASYVYLSMSYYFDRDDVALKNFAKYFLHQSHEEREHAE
KLMKLQNQRGGRIFLQDIKKPDCDDWESGLNAMECALHLEKNVNQSLLEL
HKLATDKNDPHLCDFIETHYLNEQVKAIKELGDHVTNLRKMGAPESGLAE
YLFDKHTLGDSDNESMSSQIRQNYSTDVEAAVNSLVNLYLQASYTYLSLG
FYFDRDDVALEGVSHFFRELAEEKREGYERLLKMQNQRGGRALFQDIKKP
AEDEWGKTPDAMKAAMALEKKLNQALLDLHALGSARTDPHLCDFLETHFL
DEEVKLIKKMGDHLTNLHRLGGPEAGLGEYLFERLTLKHD

The scFv heavy and light chains are represented by italics in the amino acid sequence, heavy chain underlined. The bold text in the amino acid sequence represents the CDR regions of the variable domains. The two glycine/serine linkers are indicated in lower case, the second of which runs into the sequences of the heavy and light chains of ferritin in plain text, again heavy chain sequence underlined.

FIG. 4b is a diagrammatic representation of an exemplary MT2 fusion protein. The sequence is represented by SEQ ID 2 below:

SEQ ID No: 2
LVQPGGSLRLSCAASGFTFSSFSMSWVRQAPGKGLEWVSSISGSSGTTYY
ADSVKGRFTISRDNSKNTLYLQMNSLRAEDTAVYYCAKPFPYFDYWGQGT
LVTVSSGDgssggsggASTGEIVLTQSPGTLSLSPGERATLSCRASQSVS
SSFLAWYQQKPGQAPRLLIYYASSRATGIPDRFSGSGSGTDFTLTISRLE
PEDFAVYYCQQTGRIPPTFGQGTKVEIKsgggMDPNCSCAAGDSCTCAGS
CKCKECKCTSCKKSCCSCCPVGCAKCAQGCICKGASDKCSCCAPGSAGGS
GGDSMAEVQLLE.

The scFv sequence is in italics, with heavy chain underlined, bold text highlights CDRs. The two linker sequences are in lower case, with the second running into the metallothionein sequence given in normal text.

The scFv-ferritin and scFv-MT2 fusion proteins may be expressed in strains of E. coli. This is typically achieved by transforming susceptible E. coli cells with a plasmid encoding one or other of the fusion proteins. The expression plasmids typically contain elements for bacterial translation and expression as well as enhancer sequences for increased expression. However, it is preferable that fusion proteins are expressed in mammalian expression systems.

The plasmid also preferably contains a sequence for antibiotic resistance. When the bacterial cells are spread onto an agar nutrient plate containing the antibiotic, cells that do not contain the plasmid will not divide. Those cells that do contain the plasmid are able to grow in discreet colonies. Each cell in the colony is descended from a single cell or ‘clone’ (therefore the process is known as cloning).

The clones may be picked from the plate and grown in liquid media containing antibiotic. Fusion protein expression is generally initiated by the addition of an inducer (such as isopropyl β-D-1-thiogalactopyranoside or IPTG). The cells may be incubated for a limited amount of time before being harvested. The cells may be lysed using urea, and the lysates analysed, e.g. by SDS-PAGE and Western blotting.

### Protein Detection and Purification

The protein expression profile of clones may be assessed using SDS-PAGE (sodium dodecyl sulphate polyacrylamide gel electrophoresis) and Western blotting. In these assays, proteins are chemically denatured (by severing sulphur bonds using chemicals such as β-mercaptoethanol and/or by the addition of SDS which eliminates intra-bond electro-static charges). Cell lysates are added to a well at the top of the gel. An electric current (DC) is then applied to the gel and proteins migrated through the gel according to their size. The proteins are then visualised by staining the gel with a dye. Specific proteins are probed for by transferring the separated proteins onto a nitrocellulose membrane (again by using an electric current). Specific enzyme-linked antibodies are incubated on the sheet and substrate (a colourimetric, luminescent or fluorescent chemical) is added to visualise proteins.

The clone with the highest level of expression is usually expanded and grown at large scale (1 litre). The cells are induced as above and harvested.

The harvested cells are lysed and the proteins purified using, for example, metal affinity chromatography. Other methods of purification may be employed, if desired, including fibronectin affinity columns.

Magnetisation of Ferritin and scFv-Ferritin

The iron within ferritin is not paramagnetic. The iron is usually in the form of Fe (III). In order to produce paramagnetic ferritin, the iron with ferritin (and ultimately, the fusion protein) is removed without damaging the protein; the iron was then replaced with a paramagnetic form (Fe (II)).

There are several forms of iron oxide and not all these forms are equally magnetic. E.g. FeO, Fe₂O₃ and Fe₃O₄. Iron oxide (Fe₃O₄) or ferrous ferric oxide, also known as magnetite or lodestone is the most magnetic form.

The contrast agent can be administered to the patient in any manner known in the art, e.g. by topical, enteral or parenteral administration. Examples of suitable administration methods are intravenous, subcutaneous, intramuscular or intraperitoneal injection, inhalation or ingestion.

However, the presence of a magnetic or magnetizable substance within the contrast agent also allows it to be directed to certain areas of the body using certain physical means. In particular, a device comprising an electromagnet and an element suitable for being inserted into the body can be used, e.g. a catheter with an electromagnet at one end. While the electromagnet is switched on the contrast agent will adhere to the catheter. The catheter is inserted into the body, and moved to the site of interest, for example into the region of a suspected blood clot. Once the catheter is in position the electromagnet is switched off and the contrast agent is released into the vicinity.

Similarly, the device may be used to remove or substantially remove the contrast agent from the patient's body once the imaging has taken place. In particular, this can be done where the contrast agent is bound to “free” cells, such as immune cells or small tumours, if the binding affinity/avidity is controllable (e.g. using a scFv with a higher dissociation constant), or where dialysis is used.

The present invention also provides an imaging method for obtaining one or more images of a patient by means of at least two contrast agents, wherein each contrast agent comprises a different binding moiety and/or a different recognition moiety.

In particular, the magnetic properties of each contrast agent in such a method can be distinct from the other contrast agents being used. This is achieved through each contrast agent comprising a unique combination of different magnetic or magnetizable substances and/or each having a unique quantity of a single magnetic or magnetizable substance. Typically the contrast agents each have the same magnetic substance (e.g. Fe) but present in different quantities. In some embodiments a unique combination of magnetic substances may be employed to ensure each contrast agent has a unique property (e.g. Fe and Co; Fe and Mn; Co and Mn; etc.). The combination includes sets in which each contrast agent has a single substance but each substance is different in each agent (e.g. Fe; Co; Mn; etc.).

In effect, each contrast agent comprises a different magnetic protein/polypeptide/peptide species so that each agent can be identified separately in an image taken with a magnetic field of a particular strength. Accordingly, two different contrast agents can be utilised together and their respective locations within the patient identified during one imaging session.

The present invention also relates to products for use in the imaging method. In particular, the present invention provides a contrast agent composition suitable for use in an imaging method wherein the agent comprises:
(a) a binding moiety; and
(b) a recognition moiety;
   wherein the binding moiety comprises a metal-binding protein, polypeptide or peptide which is bound to or encapsulates a magnetic or magnetizable substance, and wherein the composition optionally contains a further component suitable for use in a contrast agent composition.

In a preferred embodiment the further component is selected from an excipient, a carrier, a solvent, a diluent, an adjuvant and a buffer.

The invention will now be described in more detail, by way of example only, with reference to the following example.

### EXAMPLES

### Example 1

### In Vivo Imaging Method

It is envisaged that the method of the present invention can be used in the following way to monitor the size and spread of a solid tumour during treatment of a cancer patient.

A fusion protein comprising a ferritin binding moiety encapsulating paramagnetic particles, and a recognition moiety is generated. In particular, the recognition moiety is an scFv portion from an antibody specific for a receptor expressed on the surface of the tumour cells. Such a fusion protein can be generated by recombinant techniques that are well-known in the art.

The fusion protein, in a formulation suitable for pharmaceutical use, is injected into the patient's body in the vicinity of the tumour. After a short period, the patient is placed in the MR imager and images are gathered from the region of the body in which the tumour is situated.

The following experimental detail indicates how the fusion protein of a recognition moiety and a binding moiety can be made:

### Example 2

### Design and Manufacture of Fusion Proteins

In order to exemplify the invention, fusion proteins were designed, using commercially available murine anti-fibronectin antibody. Fusion proteins consisting of anti-fibronectin scFv genetically linked by short flexible linkers to either MT2, or ferritin were produced. This Example details the construction of the fusion proteins, their characterisation and isolation.

The design of the anti-fibronectin ferritin or MT2 fusion proteins was based on cloning the V_{H} and V_{L} genes from a mouse anti-fibronectin antibody into a vector. Both genes were linked by short, flexible linkers composed of small non-charged amino acids. Immediately at the 3′ end of the V_{L} gene, another short flexible linker led into either the ferritin genes or the MT2 gene. Both fusion proteins had a six-histidine region for purification on nickel columns. The fusion protein translation was terminated at a stop codon inserted at the 3′ end of the ferritin light gene or the MT2 gene. The plasmid vector containing all these elements was used to transform bacteria for expression.

The genes for the ferritin and MT2 were obtained from cDNA libraries. A cDNA library is formed by obtaining mRNA from cells or tissues, reverse transcribing the RNA to cDNA using an enzyme known as reverse transcriptase and cloning each individual cDNA into a plasmid vector (see FIG. 6).

### Generation of the Anti-Fibronectin: Ferritin Fusion Protein

### Background

Ferritin is a 12-nm diameter protein with a molecular weight of approximately 480 kDa. The protein consists of a large cavity (8 nm diameter) which encases iron. The cavity is formed by the spontaneous assembly of 24 ferritin polypeptides folded into four-helix bundles held by non-covalent bonds. The amino acid sequence and therefore the secondary and tertiary structures of ferritin are conserved between animals and plants. The structure of the protein in bacteria is the same as eukaryotes, although the sequence is different. Two types of subunits (heavy chain (H) and light chain (L)) form ferritin in vertebrates, each with catalytically active (H) or inactive (L) oxidase sites. The ratio of heavy and light chains varies according to requirement. The amino acid sequences of the ferritin heavy and light chains used in the construction of the fusion proteins are:

Ferritin heavy chain
(molecular weight 21096.5 Da):
(SEQ ID No: 3)
MTTASTSQVRQNYHQDSEAAINRQINLELYASYVYLSMSYYFDRDDVALK
NFAKYFLHQSHEEREHAKLMKLQNQRGGRIFLQDIKKPDCDDWESGLNAM
ECALHLEKNVNQSLLELHKLATDKNDPHLCDFIETHYLNEQVKAIKELGD
HVTNLRKMGAPESGLAEYLFDKHTLGDSDNES
Ferritin light chain
(molecular weight 20019.6 Da):
(SEQ ID No: 4)
MSSQIRQNYSTDVEAAVNSLVNLYLQASYTYLSLGFYFDRDDVALEGVSH
FFRELAEEKREGYERLLKMQNQRGGRALFQDIKKPAEDEWGKTPDAMKAA
MALEKKLNQALLDLHALGSARTDPHLCDFLETHFLDEEVKLIKKMGDHLT
NLHRLGGPEAGLGEYLFERLTLKHD

Together with the anti-fibronectin scFv amino acid sequences, the predicted sequence of a single polypeptide of the fusion protein is (with the linker sequences between the heavy and light antibody genes and between the antibody light chain and ferritin heavy chain highlighted in lower case):

(SEQ ID No: 1)
LVQPGGSLRLSCAASGFTFSSFSMSWVRQAPGKGLEWVSSISGSSGTTYY
ADSVKGRFTSRDNSKNTLYLQMNSLRAEDTAVYYCAKPFPYFDYWGQGTL
VTVSSGDgssggsggASTGEIVLTQSPGTLSLSPGERATLSCRASQSVSS
SFLAWYQQKPGQAPRLLIYYASSRATGIPDRFSGSGSGTDFTLTISRLEP
EDFAVYYCQQTGRIPPTFGQGTKVEIKsgggMTTASTSQVRQNYHQDSEA
AINRQINLELYASYVYLSMSYYFDRDDVALKNFAKYFLHQSHEEREHAKL
MKLQNQRGGRIFLQDIKKPDCDDWESGLNAMECALHLEKNVNQSLLELHK
LATDKNDPHLCDFIETHYLNEQVKAIKELGDHVTNLRKMGAPESGLAEYL
FDKHTLGDSDNESMSSQIRQNYSTDVEAAVNSLVNLYLQASYTYLSLGFY
FDRDDVALEGVSHFFRELAEEKREGYERLLKMQNQRGGRALFQDIKKPAE
DEWGKTPDAMKAAMALEKKLNQALLDLHALGSARTDPHLCDFLETHFLDE
EVKLIKKMGDHLTNLHRLGGPEAGLGEYLFERLTLKHD

The molecular weight of the polypeptide component was 65.550 kDa.

### Assembly of the Anti-Fibronectin: Ferritin Fusion Protein Genes

Ferritin heavy and light chain genes were amplified from a human liver cDNA library using PCR (see FIG. 7a). The PCR products were of the expected size (˜540 bp). These PCR products were ligated using overlapping PCR (FIG. 7b—the product is of the expected size).

The overlap PCR product was gel purified and ligated into a sequencing vector for sequencing analysis. This involved transforming bacteria with the sequencing vector containing the ferritin heavy and light chain overlapped genes. The transformed bacteria were then spread on an antibiotic containing plate to separate clones. The cells were incubated overnight to allow colonies to form. Individual colonies were then picked from the plate and grown in liquid media. The plasmids from each clone were isolated and analysed using PCR (FIG. 7c). Clone 4 was found to contain the expected sequence. The DNA from this clone was therefore subsequently used in all further work.

The variable heavy and light chain genes for a murine anti human fibronectin antibody were PCR amplified from a monoclonal hybridoma. These genes have previously been joined by a flexible linker region to form a scFv. This scFv gene fusion was amplified using PCR. The DNA gel of this amplification can be seen in FIG. 8a alongside the ferritin polygene overlap product. The relevant bands were excised from the gel and the DNA purified. This was then used in a further overlap PCR to conjugate the scFv and ferritin polygene (FIG. 8b). The arrowed band is of the expected size for the scFv:ferritin fusion. This was excised and the DNA purified for further use.

The primers used to do this contained sequences to allow for endonuclease (enzymes able to cut specific sequences of double stranded DNA) restriction of the DNA for ligation into a plasmid.

After gel purification, the scFv:ferritin PCR product was restricted using the restriction enzymes (endonucleases) Bam H1 and EcoR1. The purified restricted products were subsequently cloned into two expression vectors; pRSET and pET26b. Clones were isolated as before and the results of a PCR to identify positive clones can be seen in FIG. 9.

Colonies 3-5 and 7 from the set containing the plasmid pRSET and colony 6 from the set containing the plasmid pET26b were selected for sequence analysis.

The resulting data demonstrated that clones pRSET 4 and 5 and pET26b clone 6 contained the scFv:ferritin construct. The clone pRSET 4 was used for protein expression.

Anti-Fibronectin scFv:Ferritin Fusion Protein Expression

To validate the expression of the fusion protein, three 5 ml cultures were grown in LB broth (Luria-Bertani broth: 10 g tryptone, 5 g yeast extract, 10 g NaCl per litre). The cells were induced to express protein using IPTG (isopropyl β-D-1-thiogalactopyranoside) at varying times. The cultures were then lysed in 8M urea and analysed using SDS-PAGE. The gels were stained using Coomassie blue for protein content (results in FIG. 10). Western blots using an anti-polyhistidine antibody were performed to specifically identify the fusion protein (FIG. 10).

The time-points for induction were 2, 3 and 4 hours after inoculation.

The bands seen in the blot demonstrated that the fusion protein was being expressed and could be detected using an anti-histidine antibody. The polypeptide was approximately 75-85 kDa in size. The expression yields were relatively high and over-expression was evident as the fusion protein bands correspond to the very dark bands seen in the Coomassie blue stained gel. Inducing 3 hours after inoculation gave relatively high levels of expression and was used for subsequent expression.

### Generation of the Anti-Fibronectin:MT2 Fusion Protein

### Background

Metallothioneins are intracellular, low molecular weight, cysteine-rich proteins. These proteins are found in all eukaryotes and have potent metal-binding and redox capabilities. MT-1 and MT-2 are rapidly induced in the liver by a variety of metals, drugs and inflammatory mediators. MT2 binds seven divalent transition metals via two metal binding clusters at the carboxyl (α-domain) and amino (β-domain) terminals. Twenty cysteine residues are involved in the binding process.

The sequence of MT2 is:

(SEQ ID No: 5)
MDPNCSCAAGDSCTCAGSCKCKECKCTSCKKSCCSCCPVGCAKCAQGCIC
KGASDKCSCCAPGSAGGSGGDSMAEVQLLE

Together with the anti-fibronectin scFv amino acid sequences, the predicted sequence of a single polypeptide of the fusion protein is (with the linker sequences between the heavy and light antibody genes and between the antibody light chain and MT2 heavy chain highlighted in lower case):

(SEQ ID No: 2)
LVQPGGSLRLSCAASGFTFSSFSMSWVRQAPGKGLEWVSSISGSSGTTYY
ADSVKGRFTISRDNSKNTLYLQMNSLRAEDTAVYYCAKPFPYFDYWGQGT
LVTVSSGDgssggsggASTGEIVLTQSPGTLSLSPGERATLSCRASQSVS
SSFLAWYQQKPGQAPRLLIYYASSRATGIPDRFSGSGSGTDFTLTISRLE
PEDFAVYYCQQTGRIPPTFGQGTKVEIKsgggMDPNCSCAAGDSCTCAGS
CKCKECKCTSCKKSCCSCCPVGCAKCAQGCICKGASDKCSCCAPGSAGGS
GGDSMAEVQLLE

### Assembly of the Anti-Fibronectin:MT2 Fusion Protein Genes

The metallothionein II genes were amplified from a human liver cDNA library using PCR (FIG. 11). The PCR products were of the expected size (˜200 bp).

The PCR product was restricted using the Bgl II restriction enzyme and ligated into a previously cut plasmid (Factor Xa vector).

Colony PCR of selected clones revealed bands for all clones selected (FIG. 12). Clones 2, 4 and 9 were selected for sequencing analysis. Clone 9 was used in further work.

Anti-Fibronectin scFv:MT2 Fusion Protein Expression

To validate expression of the scFv:MT2 fusion protein, three 5 ml cultures were grown in LB broth induced (IPTG) at different time-points as with the ferritin fusion protein. The cultures were lysed in 8M urea and analysed using SDS-PAGE gels stained with Coomassie blue and blotted using an anti-histidine antibody (FIG. 13). Cells induced 4 hours after inoculation produced slightly more protein (lane 3 on both gels). These growth conditions were used for subsequent protein expression.

### Purification of Fusion Proteins

The isolation of soluble protein by isolating, washing and re-solubilising inclusion bodies was employed.

The protocol takes approximately one week to complete. Photographs of a Coomassie blue stained gel and western blot of the re-solubilised scFv:ferritin and scFv:MT2 fusion proteins can be seen in FIG. 14. The fusion proteins are circled—ferritin is in lane 2 on both gels and MT2 is in lane 3 of both gels. A protein molecular weight ladder is in lane 1.

From this, it can be seen that the fusion proteins were successfully expressed and concentrated. These proteins were be used in magnetising protocols and further experiments.

### Example 3

### SPR Analysis

Anti-fibronectin ferritin and MT2 fusion protein inclusion body preparations were used in surface plasmon resonance (SPR) assays using a SensiQ instrument (ICX Nomadics).

For these experiments, a fibronectin peptide was coupled to the surface of a carboxyl chip. The fusion protein preps were then flowed over the chip and association (Kₐ) and dissociation kinetics (K_{d}) determined

### Fusion Protein Samples for Analysis

Six samples of each fusion protein, with varying concentration from 0.0013-0.133 μM were produced in running buffer as set out in Table 2 and Table 3 below.

TABLE 2
Metallothionein Fusion Protein 75 kDa:
40 μl 100 μg/ml 75 kDa/360 μl running buffer
to give 400 μl 10 μg/ml (0.133 μM) then:

| μM FP | μg/ml FP | μl of 10 μg/ml FP | μl of running buffer |
|---|---|---|---|
| 0.0013 | 0.1 | 20 (of 1 μg/ml) | 180 |
| 0.0065 | 0.5 | 10 | 190 |
| 0.013 | 1 | 20 | 180 |
| 0.05 | 3.75 | 75 | 125 |
| 0.1 | 7.5 | 150 | 50 |
| 0.133 | 10 | 400 | 0 |

TABLE 3
Ferritin ED-B Fusion Protein 270 kDa:
144 μl 100 μg/ml 270 kDa/256 μl running buffer
to give 400 μl 36 μg/ml (0.133 μM) then:

| μM FP | μg/ml FP | μl of 36 μg/ml FP | μl of running buffer |
|---|---|---|---|
| 0.0013 | 0.36 | 20 (of 3.6 μg/ml) | 180 |
| 0.0065 | 1.8 | 10 | 190 |
| 0.013 | 3.6 | 20 | 180 |
| 0.05 | 9 | 75 | 125 |
| 0.1 | 18 | 150 | 50 |
| 0.133 | 36 | 400 | 0 |

### Metallothionein

### Sample (Cycles 1-6)=20 μl 0.0013-0.133 μM Metallothionein Fusion Protein

Assay run=MAb & Gly assay cycle (as above)

Sensograms from the above cycles were overlaid using the SensiQ Qdat analysis software, and a model fitted to the data to calculate kinetic parameters (Kₐ, K_{d}). The best estimate of the K_{d} was achieved by fitting a model to just the dissociation part of the data. The result is shown in FIG. 15a. This relates to a Kₐ of 0.00503 s⁻¹ to give a K_{d} of 2.289×10⁻⁹ M (Kₐ 2.197×10⁶M⁻¹s⁻¹)

### Ferritin

### Sample (Cycles 1-6)=20 μl 0.0013-0.133 μM Ferritin Fusion Protein

Assay run=MAb & Gly assay cycle (as above)

Sensograms from the above cycles were overlaid using the SensiQ Qdat analysis software and a model fitted to the data to calculate kinetic parameters (Kₐ, K_{d}). The best estimate of the K_{d} was achieved by fitting a model to just the dissociation part of the data. The result is shown in FIG. 15b. This relates to a K_{d} of 0.00535 s⁻¹ to give a K_{d} of 6.538×10⁻¹⁰ M (Kₐ 8.183×10⁶M⁻¹s⁻¹).

### Results

From the above experimental data, it was determined that fibronectin extra domain B (aa 16-42) antigen was successfully coated onto the SensiQ chip As expected, both the 75 kDa Metallothionein Fusion Protein and the 270 kDa Ferritin Fusion Protein recognised and bound to the antigen in a specific manner. Kinetic data on the interactions of the fusion proteins with the antigen were estimated and were found to be similar and in the expected range for both fusion proteins i.e. K_{d}s in the 10⁻⁹ M range compared to 10⁻⁸ M to 10⁻¹⁰ M for most antibody/antigen interactions.

Thus, the values obtained using this instrument suggest binding affinities which compare favourably with the binding affinities of relatively high affinity antibodies. In addition, the data obtained suggest that the fusion proteins have multiple binding sites for antigen. This was expected for the ferritin fusion protein. However, this was not expected for the MT2 fusion protein and would suggest that the fusion protein is forming dimers or higher order multimeric proteins which would increase the avidity of binding.

### Example 4

### Magnetising Ferritin

Ferritin normally contains hydrated iron (III) oxide. In order to produce paramagnetic ferritin, these ions were replaced with magnetite (Fe₃O₄) which has stronger magnetic properties. The method used for this experiment involved the addition to apoferritin of iron ions and oxidation of these ions under controlled conditions.

### Materials

Reverse osmosis water (RO water)
50 mM N-(1,1-Dimethyl-2-hydroxyethyl)-3-amino-2-hydroxypropanesulfonic acid (AMPSO) buffer pH8.6 (Sigma A6659)
0.1M Sodium acetate buffer pH4.5
Phosphate buffered saline (PBS) 10 mM phosphate, 140 mM NaCl pH 7.4
Trimethylamine-N-oxide (TMA) (Sigma 317594)
0.1M ammonium iron (II) sulphate
Horse spleen apoferritin (Sigma A3641)

### Method

Trimethylamine-N-oxide (TMA) was heated in an oven to 80° C. for 30 minutes to remove Me₃N before cooling to room temperature. 114 mg TMA was added to 15 ml RO water to produce a 0.07M solution. The iron and TMA solutions were purged with N₂ for 15 minutes before use.

AMPSO buffer (1 litre) was de-aerated with N₂ for an hour. 3.0 ml apoferritin (66 mg/ml) was added to the AMPSO buffer and the solution de-aerated for a further 30 minutes. The AMPSO/apoferritin solution in a 1 litre vessel was placed into a preheated 65° C. water bath. The N₂ supply line was removed from within the solution and suspended above the surface of the solution to keep the solution under anaerobic conditions. The initial addition of iron ammonium sulphate scavenges any residual oxygen ions that may be in the solution.

Aliquots of the 0.1M iron ammonium sulphate and TMA buffers were added every 15 minutes as follows:

1^{st} addition 600 μl 0.1M iron ammonium sulphate
2^{nd} addition 600 μl 0.1M iron ammonium sulphate and 400 μl TMA
3^{rd} addition 600 μl 0.1M iron ammonium sulphate and 400 μl TMA
4^{th} addition 600 μl 0.1M iron ammonium sulphate and 400 μl TMA
5^{th} addition 900 μl 0.1M iron ammonium sulphate and 600 μl TMA
6^{th} addition 900 μl 0.1M iron ammonium sulphate and 600 μl TMA
7^{th} addition 900 μl 0.1M iron ammonium sulphate and 600 μl TMA
8^{th} addition 900 μl 0.1M iron ammonium sulphate and 600 μl TMA

Upon the latter additions of Fe and TMA, the solution colour changed from a straw colour to dark brown with dark particulates dispersed throughout. This solution is termed “magnetoferritin” from this point onwards.

The magnetoferritin solution was incubated at room temperature overnight with a strong neodymium ring magnet held against the bottle. The following day, dark solid material had been drawn towards the magnet as can be seen in the photographs in FIG. 16.

### Concentration of Magnetoferritin

Five hundred millilitres of the magnetoferritin solution was passed through 5 Macs® LS columns on magnets (with approximately 100 ml magnetoferritin passing through each column) The solution which flowed through the columns (termed ‘flow-through’) was collected in Duran bottles. The captured material from each column was eluted using 3 ml PBS by removing the columns from the magnets, adding the 3 mls PBS and using the supplied plunger resulting in approximately 4.5 ml from each column. Approximately 1 ml was stored at 2-8° C. for later analysis (termed ‘pre-dialysis concentrated magnetoferritin’). The remainder of the eluted solution (˜20 ml) was dialysed (termed ‘post-dialysis concentrated magnetoferritin’) against 5 litres of PBS at 4° C. overnight to remove excess Fe and TMA. The change in colour of the solution was noted. The original magnetoferritin was dark brown, the flow through straw coloured and the Macs® column concentrated material dark brown to black.

Dialysis tubing (Medicell International Ltd. Molecular weight cut-off 12-14000 Daltons ˜15 cm) was incubated in RO water for ten minutes to soften the tubing. The magnetically isolated concentrated magnetoferritin was transferred to the dialysis tube and incubated in 5 litres PBS at 2-8° C. with stirring overnight. The PBS solution was refreshed three times the following day at two hour intervals with dialysis continuing at 2-8° C.

### Analysis of Magnetoferritin

In order to compare the amount of magnetic protein isolated using the magnet, enzyme linked immunosorbant assay (ELISA) analysis was performed.

### Materials

Carbonate Buffer (0.159 g sodium carbonate and 0.3 g sodium bicarbonate in 100 mls RO water).
Phosphate buffered saline (PBS) 10 mM phosphate, 140 mM NaCl pH 7.4
1% bovine serum albumin (BSA (Celliance 82-045-2)) in PBS
Horse spleen apoferritin (Sigma Aldrich A3641)
Rabbit anti-horse ferritin antibody (Sigma Aldrich F6136)
Goat anti-rabbit antibody (Sigma A3687)
Substrate liquid stable phenolphthalein phosphate
Stop Solution (212 g sodium carbonate, 110.5 g 3-(cyclohexylamino)-1-propanesulfonic acid (CAPS), 217 g ethylenediamine tetraacetic acid (EDTA) 80 g sodium hydroxide, water to 5 litres)
Maxisorb Microtitre plate (NUNC Cat: 468667)

### Method

Dilutions of apoferritin were made (50 μg/ml, 25 μg/ml, 12.5 μg/ml, 6.25 μg/ml, 3.125 μg/ml and 1.5625 μg/ml) for quantification of the magnetoferritin.

The magnetoferritin (unpurified), concentrated pre-dialysis, post-dialysis and flow through was diluted in carbonate buffer at the following dilutions:

Magnetoferritin, pre-dialysis and post-dialysis dilutions:
100, 200, 400, 800, 1600, 3200, 6400 and 12800 fold dilution.

### Flow-through:

10, 20, 40, 80, 160, 320, 640 and 1280 fold dilution.

100 μl of each solution was added to wells of a microtitre plate in duplicate. Carbonate buffer (100 μl) was added to two wells as a negative control. The plate was incubated overnight at 4° C. The next day, the solution was flicked off and the wells blocked using 200 μl 1% BSA at room temperature for an hour. After washing three times with 300 μl PBS per well, the wells were patted dry before the addition of 100 μl 10 μg/ml anti-horse ferritin antibody. This was incubated for an hour at room temperature before being removed and wells washed as before.

AP-conjugated anti rabbit antibody was diluted 1 in 3500 in PBS to give a concentration of 7.43 μg/ml and incubated at room temperature for an hour. The antibody conjugate was removed and wells washed as before. AP substrate (100 μl) was added to each well and allowed to develop for 15 minutes before the addition of stop solution. Absorbances were recorded using a Varioskan Flash instrument (Thermo Fisher).

The Macs® columns retained over 35 times the amount of magnetoferritin found in the flow through indicating that magnetisation of the protein had been successful.

### Production of Apoferritin/Demineralisation of Horse Spleen Ferritin.

### Materials

0.1M sodium acetate buffer pH 4.5
Thioglycolic acid (Sigma T6750)
Horse Spleen Ferritin (Sigma 96701)
Phosphate buffered saline (PBS) 10 mM phosphate, 140 mM NaCl pH 7.4

### Method

Dialysis tubing was softened in RO water for 10 minutes. 10 ml 0.1M sodium acetate buffer was added to 1 ml Horse Spleen Ferritin (125 mg/ml) in the dialysis tubing which was clipped at both ends. The dialysis bag was transferred to 0.1M sodium acetate buffer (˜800 ml) which had been purged with N₂ for one hour. Thioglycolic acid (2 ml) was added to the buffer and N₂ purging was continued for two hours. A further 1 ml thioglycolic acid was added to the sodium acetate buffer followed by another thirty minutes of N₂ purging. The sodium acetate buffer (800 ml) was refreshed and purging continued. The demineralisation procedure was repeated until the ferritin solution was colourless. The N₂ purge was stopped and the apoferritin solution was dialysed against PBS (2 L) for 1 h with stirring. The PBS was refreshed (3 litres) and the apoferritin solution was dialysed in PBS at 2-8° C. overnight.

### Results

The ferritin solution changed colour during the procedure from light brown to colourless indicating removal of iron.

### Analysis of Heat Treatment on the Anti-Fibronectin:Ferritin Fusion Protein

### Materials

Carbonate buffer (0.159 g sodium carbonate, 03 g sodium bicarbonate in 100 mls water)
Phosphate buffered saline (PBS)
1% bovine serum albumin (BSA (Celliance 82-045-2)) in PBS
Fibronectin peptide
Anti-fibronectin:ferritin fusion protein (scFv:ferritin)
Anti-human ferritin murine monoclonal antibody (Santa Cruz SC51887)
Anti-mouse alkaline phosphatase antibody (Sigma A3562)
Substrate liquid stable phenolphthalein phosphate
Stop Solution (212 g sodium carbonate, 110.5 g 3-(cyclohexylamino)-1-propanesulfonic acid (CAPS), 217 g ethylenediamine tetraacetic acid (EDTA) 80 g sodium hydroxide, water to 5 litres)
Maxisorb Microtitre plate (NUNC Cat: 468667)

### Method

100 μl (at 100 μg/ml) scFv:ferritin was transferred to a thin walled PCR tube and heated in a thermocycler at 60° C. for 30 minutes.

Wells of a microtitre plate were coated with fibronectin peptide (supplied at 1.5 mg/ml) diluted in carbonate buffer to 15 μg/ml and incubated overnight at 4° C. Excess solution was flicked off and the plate blocked using 1% BSA in PBS for 1 hour at room temperature. This was flicked off and the plate washed three times using PBS. The scFv:ferritin fusion protein and heat treated scFv:ferritin fusion protein were added to wells at a concentration of 33 μg/ml (100 μl each). The ferritin fusion proteins were incubated for 2 hours at room temperature before being removed and the wells washed as before. Mouse anti-ferritin antibody was added at a concentration of 20 μg/ml and added at a volume of 100 μl to each well and incubated at room temperature for an hour. This was removed and the wells washed as before. Goat anti-mouse AP conjugated antibody was diluted (50 μl+950 μl PBS) and added at a volume of 100 μl to all wells. This was incubated at room temperature for an hour and removed as before. Substrate was added to all wells and incubated at room temperature for 45 minutes and the reaction stopped using stop buffer. Absorbances were recorded using a Varioskan Flash instrument (Thermo Fisher Electron).

The scFv:ferritin retains binding ability to fibronectin and remains detectable by the anti-human ferritin monoclonal antibody after heating to 60° C. for 30 minutes (FIG. 18).

### Anti Fibronectin:Ferritin Fusion Protein Demineralisation

### Materials

Anti-fibronectin:ferritin fusion protein (scFv:ferritin).
0.1M sodium acetate buffer
Thioglycolic acid (70% w/w Sigma T6750)
Phosphate buffered saline (PBS) 10 mM phosphate, 140 mM NaCl pH 7.4

### Method

The scFv:ferritin fusion protein was thawed from −20° C. to room temperature. Nine millilitres of 100 μg/ml was dispensed into softened dialysis tubing. The tubes which had contained the fusion protein were rinsed with a total of 1 ml sodium acetate buffer which was added to the 9 ml of protein (to give a 0.9 mg/ml solution). 800 ml sodium acetate buffer was purged with N₂ for 15 minutes before the dialysis bag was added. The solution was then purged for a further 2 hours. 2 ml thioglycolic acid was added to the buffer which continued to be purged using N₂. After a further 2 hours, another 1 ml of thioglycolic acid was added. The buffer was refreshed (800 ml pre-purged sodium acetate buffer containing 3 ml thioglycolic acid) and dialysis continued under N₂ for 1 hour. The dialysis bag was then transferred to 2 litres PBS at room temp (no N₂) then overnight at 4° C. in 3 litres PBS. This demineralised fusion protein was then used to produce paramagnetic fusion protein by the addition of iron and controlled oxidation as below.

Production of Magnetic scFv:Ferritin

### Materials

Reverse osmosis water (RO water)
50 mM N-(1,1-Dimethyl-2-hydroxyethyl)-3-amino-2-hydroxypropanesulfonic acid (AMPSO) buffer pH8.6 (Sigma A6659)
0.1M Sodium acetate buffer pH4.5
Phosphate buffered saline (PBS) 10 mM phosphate, 140 mM NaCl pH 7.4
Trimethylamine-N-oxide (TMA) (Sigma 317594)
0.1M ammonium iron (II) sulphate

Trimethylamine-N-oxide (TMA) was heated in an oven to 80° C. for 30 minutes to remove Me₃N before cooling to room temperature. 114 mg TMA was added to 15 ml RO water to produce a 0.07M solution. The iron and TMA solutions were purged with N₂ for 15 minutes before use.

The demineralised fusion protein contained within a dialysis bag (detailed above) was dialysed against 1 litre AMPSO buffer for 2 hour at room temp with stirring under nitrogen. The demineralised scFv:ferritin (˜10 ml) was transferred to a conical flask. 18 μl iron solution was added to the demineralised protein solution whilst purging with N₂ to scavenge any residual oxygen. After 25 minutes, 15 μl iron and 10 μl TMA were added.

The following further amounts of iron and TMA buffers were then added at 15 minute intervals:

3^{rd} addition: 30 μl iron+20 μl TMA.
4^{th} addition: 15 μl iron+10 μl TMA
5^{th} addition: 15 μl iron+10 μl TMA
6^{th} addition: 15 μl iron+10 μl TMA

The magnetised protein was passed through a Macs® LS column. The flow through was passed though a second time to try and increase capture efficiency. The magnetised protein was eluted from the column by removing the column from the magnet and adding 1 ml PBS and using the plunger (eluate approx 2 ml). This represents a two-fold dilution of the protein on the column.

Eluted protein and controls were coated onto a microtitre plate for analysis as detailed below.

Analysis of the scFv:Magnetoferritin Fusion Protein by ELISA

In order to ascertain if the magnetised fusion protein retains binding to an anti-ferritin monoclonal antibody an enzyme linked immunosorbant assay was performed.

### Materials

Carbonate buffer (0.159 g sodium carbonate, 03 g sodium bicarbonate in 100 mls water) pH 9.6
Phosphate buffered saline (PBS) 10 mM phosphate, 140 mM NaCl pH 7.4
Fibronectin peptide
Anti-fibronectin:ferritin fusion protein (scFv:ferritin)
Anti-human ferritin murine monoclonal antibody (Santa Cruz SC51887)
Anti-mouse alkaline phosphatase antibody (Sigma A3562)
Substrate liquid stable phenolphthalein phosphate
Stop Solution (212 g sodium carbonate, 110.5 g 3-(cyclohexylamino)-1-propanesulfonic acid (CAPS), 217 g ethylenediamine tetraacetic acid (EDTA) 80 g sodium hydroxide, water to 5 litres)
Maxisorb Microtitre plate (NUNC Cat: 468667)

### Method

### Fusion Protein Coated Wells

Wells were coated with scFv:ferritin (untouched), scFv:magnetoferritin, scFv:magnetoferritin eluted from the Macs® column and the flow through at a concentration of 1 n 3 in carbonate buffer. The plate was incubated over a weekend at 4° C. Excess solution was flicked off and the plate blocked using 1% BSA in PBS for 1 hour at room temperature. This was flicked off and the plate washed three times using PBS (300 μl/well for each wash). Mouse anti-ferritin antibody was added at a concentration of 20 μg/ml and added at a volume of 100 μl to each well and incubated at room temperature for an hour. This was removed and the wells washed as before. Goat anti-mouse AP conjugated antibody was diluted to 10 μg/ml and added at a volume of 100 μl to all wells. This was incubated at room temperature for an hour and removed as before. Substrate was added to all wells and incubated at room temperature for an hour and the reaction stopped using stop buffer. Absorbances were recorded using a Varioskan Flash instrument (Thermo Fisher Electron) (see FIG. 19a).

### Fibronectin Coated Wells

Wells of a microtitre plate were coated with 100 μl fibronectin peptide (supplied at 1.5 mg/ml) diluted in carbonate buffer to 15 μg/ml. The plate was incubated overnight at 2-8° C. Excess solution was flicked off and the wells washed three times in 300 μl PBS. The scFv:ferritin fusion proteins were added neat to the appropriate wells (100 μl) in duplicate. The plate was then incubated for an hour at room temperature. The solution was flicked off and the wells washed three times in 300 μl PBS. Mouse anti-ferritin antibody was added at a concentration of 20 μg/ml and added at a volume of 100 μl to each well and incubated at room temperature for an hour. This was removed and the wells washed as before. Goat anti-mouse AP conjugated antibody was diluted to 10 μg/ml and added at a volume of 100 μl to all wells. This was incubated at room temperature for an hour and removed as before. Substrate was added to all wells and incubated at room temperature for 45 minutes and the reaction stopped using stop buffer. Absorbances were recorded using a Varioskan Flash instrument (Thermo Fisher Electron) (see FIG. 19b).

The Macs® columns have concentrated the magnetised fusion protein and it is still recognised by the monoclonal anti-ferritin antibody, indicating that the anti-fibronectin-ferritin fusion protein has been magnetised and retained structural integrity. The data also indicates that the magnetised anti-fibronectin ferritin fusion protein retains binding ability to its target antigen and thus illustrates a bi-functional single chain fusion protein that is both magnetizable and can bind a target selectively.

### Example 5

### Isolation of Platelets and FACS Analysis

An experiment was conducted to demonstrate the ability of the anti-fibronectin:ferritin fusion protein (scFv:ferritin) to select platelets expressing fibronectin from other cell types.

Plasma from a sample of blood which had been stored in an EDTA vacutainer at 4° C. for three days to allow most cells to settle was exposed to air for 30 minutes to activate platelets. 10 μl of this was mixed with 100 μl magnetised scFv:ferritin as described above. The magnetic fusion protein/plasma mix was incubated at room temperature for 30 minutes (10 μl was retained for analysis) before being passed through a magnetised, pre-equilibrated LS MACS column (Miltenyi Biotec). The flow through was retained for analysis. The bound fraction was eluted from the column using the supplied plunger. The fractions were diluted to 500 μl in PBS and analysed using forward and side scatter by fluorescence activated cell sorting (FACS).

The results are shown in Table 4. It should be recognised that with FACS analysis the sample is analysed until a set number of events (e.g. 10,000) have been recorded. Thus, the volume sampled can vary enormously dependent on the concentration of cells. This is particularly important when one is comparing samples with high cellular concentrations with samples where many of the cells have been removed. When calculating the efficiency of cellular removal or isolation procedure, it is necessary to correct for this change of sample volume. This is done within Table 4.

TABLE 4
Number of FACS cell type related events and selectivity
and isolation efficiency of isolating platelets from plasma.

| | | | Flow | Captured |
|---|---|---|---|---|
| Total Events | 10000 | Plasma | Though | (magnetised) |
| No of non-lymphocytes | | 9819 | 8476 | 9982 |
| No of Lymphocytes | | 181 | 1524 | 18 |
| Non-lymphocyte:lymphocyte | | 54.2 | 5.6 | 554.6 |
| ratio | | | | |
| Relative volume | | 1.0 | 8.4 | 0.1 |
| Selectivity efficiency | 99.7 | | | |
| (non-lymphocytes/lymphocytes) | | | | |
| Isolation efficiency (% of | 90.1 | | | |
| platelets captured) | | | | |

It can be seen that 90% of available platelets were captured with this un-optimised procedure with selectivity over lymphocytes of almost 100%. This demonstrates the ability of the scFv:ferritin protein to bind fibronectin displayed on the surface of platelets.

Visual inspection with a microscope (results not shown) correlated with the FACS analysis showing that fusion protein binds to platelets leading to the formation of large, granular aggregates.

### Example 6

### Further Protocols

Magnetisation of scFv MT2 Fusion Protein

The scFv-MT2 fusion proteins may be magnetised by replacing zinc ions with manganese and cadmium ions. Methods to do this may be optimised as required. The methods to achieve this include the depletion of zinc by dialysis followed by replacement, also using dialysis with adaptations of published protocols if required.

In detail, these protocols are as follows:
1. Dissolve 5 mg MT2 in 5 ml buffer (4.5M urea, 10 mM Tris base, 0.1M dithiothreitol (DTT), 0.1% mannitol and 0.5 mM Pefabloc, pH 11) to strip the protein of the metal ions.
2. Dialyse in the same buffer for 1 hours.
3. Refold the protein by dialysing in buffer 1 (10 mM tris base, 2M urea, 0.1M DTT, 0.1% mannitol, 0.5 uM Pefabloc and 1 mM Cd²⁺/Mn²⁺ pH 11) for 72 hours.
4. Change dialysis buffer to buffer 2 (as above but with urea at a concentration of 1M) and dialyse for 24 hours.
5. Change the dialysis buffer to a buffer as above containing no urea. Dialyse for 24 hours.
6. Change the dialysis buffer as in step 5 to a buffer with pH 8.8. Dialyse for 24 hours.
7. Change the buffer as in step 6 to a buffer containing no mannitol and dialyse as before.
8. Change the buffer as in step 7 to a buffer containing no Cd²⁺/Mn²⁺ and dialyse for 24 hours.

The binding characteristics may be assessed as above in Example 3 for the ferritin fusion protein.

1. An imaging method for obtaining an image of a patient by means of a contrast agent wherein the method comprises subjecting the patient to an imaging method for which the contrast agent is suitable, wherein the contrast agent comprises:
(a) a binding moiety; and
(b) a recognition moiety capable of targeting the contrast agent to a site within the body of the patient,
and wherein the binding moiety comprises a metal-binding protein, polypeptide or peptide which is bound to or encapsulates a magnetic or magnetizable substance.

2. An imaging method according to claim 1 which further comprises the step of administering the contrast agent to the patient.

3. An imaging method according to claim 1 or claim 2 which is a magnetic resonance imaging method, a nuclear magnetic resonance method or an electron spin resonance method.

4. An imaging method according to claim 1, wherein the magnetic or magnetizable substance is a paramagnetic substance.

5. An imaging method according to claim 1, wherein the contrast agent contains a fusion protein comprising the binding moiety and the recognition agent.

6. An imaging method according to claim 1, wherein the binding moiety of the contrast agent comprises a protein, or a metal-binding domain of a protein, selected from lactoferrin, transferrin, ferritin, a ferric binding protein, frataxin, a siderophore and a metallothionein.

7. An imaging method according to claim 1, wherein the magnetic or magnetizable substance of the contrast agent are transition and/or lanthanide metal atoms and/or ions and/or a compound comprising such ions.

8. An imaging method according to claim 7 wherein the transition metal and/or lanthanide ions comprise any one or more ions of Fe, Co, Ni, Mn, Cr, Cu, Zn, Cd, Y, Gd, Dy, or Eu.

9. An imaging method according to claim 8 wherein the one or more metal ions comprise any one or more of Fe²⁺, Fe³⁺, Co²⁺, Co³⁺, Mn²⁺, Mn³⁺, Mn⁴⁺, Ni²⁺, Zn²⁺, Gd³⁺ and Cd²⁺.

10. An imaging method according to claim 1, wherein the recognition moiety of the contrast agent is capable of binding a target selected from the group consisting of a cell or cellular component, a cardiovascular plaque, a neurological plaque, an area undergoing angiogenesis, an area undergoing apoptosis and a thrombi.

11. An imaging method according to claim 1, wherein the recognition moiety of the contrast agent is selected from the group consisting of an antibody or a fragment of an antibody, a fragment of a receptor, a protein, a polypeptide, a nucleic acid, and an aptamer.

12. An imaging method according to claim 11 wherein the recognition moiety is selected from a variable polypeptide chain of an antibody (Fv), a T-cell receptor or a fragment of a T-cell receptor, avidin, streptavidin, and heparin.

13. An imaging method according to claim 12 wherein the recognition moiety is selected from a single chain of a variable portion of an antibody (sc-Fv).

14. An imaging method according to claim 1, which method utilizes two or more contrast agents and wherein each contrast agent has a magnetic property that differs from every other contrast agent being utilized.

15. A contrast agent composition suitable for use in an imaging method wherein the agent comprises:
(a) a binding moiety; and
(b) a recognition moiety;
wherein the binding moiety comprises a metal-binding protein, polypeptide or peptide which is bound to or encapsulates a magnetic or magnetizable substance, and wherein the composition optionally contains a further component suitable for use in a contrast agent composition.

16. A contrast agent composition according to claim 15 wherein the further component is selected from an excipient, a carrier, a solvent, a diluent, an adjuvant and a buffer.

17. A contrast agent composition according to claim 15 or claim 16 wherein the recognition moiety targets the contrast agent to a site within the body of the patient.

18. A method for obtaining an image of a patient comprising contacting the patient with a contrast agent and imaging the patient with a suitable imager, wherein the contrast agent comprises:
(a) a binding moiety; and
(b) a recognition moiety;
and wherein the binding moiety comprises a metal-binding protein, polypeptide or peptide which is bound to or encapsulates a magnetic or magnetizable substance.

19. The method according to claim 18, wherein the recognition moiety targets the contrast agent to a site within the body of the patient.


SEQUENCE LISTING (5 sequences)

SEQ ID NO: 1 - protein, 590 aa, Artificial Sequence [sequence omitted]

SEQ ID NO: 2 - protein, 312 aa, Artificial Sequence
  scFv-MT2 Fusion Protein
LVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASTGEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKSGGGMDPNCSCAAGDSCTCAGSCKCKECKCTSCKKSCCSCCPVGCAKCAQGCICKGASDKCSCCAPGSAGGSGGDSMAEVQLLE

SEQ ID NO: 3 - protein, 182 aa, Artificial Sequence
  Ferritin Heavy Chain
MTTASTSQVRQNYHQDSEAAINRQINLELYASYVYLSMSYYFDRDDVALKNFAKYFLHQSHEEREHAKLMKLQNQRGGRIFLQDIKKPDCDDWESGLNAMECALHLEKNVNQSLLELHKLATDKNDPHLCDFIETHYLNEQVKAIKELGDHVTNLRKMGAPESGLAEYLFDKHTLGDSDNES

SEQ ID NO: 4 - protein, 175 aa, Artificial Sequence
  Ferritin Light Chain
MSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLKHD

SEQ ID NO: 5 - protein, 80 aa, Artificial Sequence
  MT2
MDPNCSCAAGDSCTCAGSCKCKECKCTSCKKSCCSCCPVGCAKCAQGCICKGASDKCSCCAPGSAGGSGGDSMAEVQLLE